(19) United States
(12) Patent Application Publication
(10) Pub. No.: US 2023/0218510 A1
(43) Pub. Date: Jul. 13, 2023

(54) LIQUID OPHTHALMIC COMPOSITION COMPRISING A SPIRULINA PLATENSIS FULL EXTRACT

(71) Applicant: WELIFEPHARMA SAGL, Lugano (CH)

(72) Inventors: Pasqua ORESTE, Milano (MI) (IT); Giorgio ZOPPETTI, Milano (MI) (IT)

(21) Appl. No.: 17/928,993

(22) PCT Filed: Jun. 2, 2021
(86) PCT No.: PCT/EP2021/064875

§ 371 Date: Dec. 1, 2022

(30) Foreign Application Priority Data
Jun. 3, 2020 (IT) 102020000013156

(51) Int. Cl. A61K 9/00; A61K 9/08; A61K 47/40; A61K 35/748; A61K 47/02; A61K 47/38

(52) U.S. Cl. CPC A61K 9/0048; A61K 9/08; A61K 47/40; A61K 35/748; A61K 47/02; A61K 47/38

(57) ABSTRACT

The present invention relates to a clear liquid ophthalmic composition comprising as the active ingredient a Spirulina platensis full extract and suitable liquid carriers and solubilising excipients. The invention also relates to a viscous liquid composition comprising an aqueous extract of spirulina platensis and to the use of said compositions in the treatment of several eye diseases.

### SUMMARY

The present invention relates to a clear liquid ophthalmic composition comprising as the active ingredient a Spirulina platensis full extract and suitable liquid carriers and solubilising excipients. The invention also relates to a viscous liquid composition comprising an aqueous extract of Spirulina platensis and to the use of said compositions in the treatment of several eye diseases.

### BACKGROUND OF THE INVENTION

Spirulina platensis or Arthrospira platensis, also referred to as Spirulina, is a microorganism belonging to the cyanobacterium class that, in the wild, live in suite water lacs with alkaline and hot water. Since ancient time it is known by people living in tropical regions for its numerous beneficial properties.

Spirulina platensis, is constituted by proteins (55-70%), carbohydrates (including the polysaccharide fraction, 15-25%) and lipids (18%), all components calculated on the material dry base. The Spirulina platensis contains also several vitamins, amino acids and minerals. Within the protein fraction there is the constituent responsible of the typical light blue colour, present at 11% on the dry base: the phycocyanin. This molecule is reported to have many beneficial properties for the human body, in particular due to its property to stimulate the immune system and to the high antioxidant power.

The Spirulina algae has been studied in vitro for its antiviral activity against HIV and other pathogens, and as chelating agent with antiproliferative function. In particular, it is known that the polysaccharide extract obtained from the microorganism has several biological properties and the healing capability were studied. For example as an antitumoral, (Mathew B. et al. “Evaluation of chemoprevention of oral cancer with Spirulina fusiformis” J. of Nutrition and Cancer 1995; 24(2), pages 197-202), as a radioprotective agent (Martinez E. et al. “Subchronic toxicity study in mice fed Spirulina” J. of Ethnopharmacology 1998; 62(3), pages 235-191) and as a hypocholesterolemic agent (Ramamoorthy A. and Premakumari S. “Effect of supplementation of Spirulina on hypercholesterolemic patients” J. of Food Science and Technology 1996; 33(2), pages 119-128).

CN101444480 describes the use of the polysaccharide fraction isolated from Spirulina platensis in the treatment of neovascular diseases in the eye, in particular against the neoangiogenesis. The polysaccharides are formulated as eye drops, at concentration of 10-500 μg/ml, preferably of 100 μg/ml.

CN103040735 describes and claims the use of the polysaccharide fraction isolated from Spirulina platensis in the treatment and prevention of bacterial keratitis in the eye. In this document the polysaccharides are formulated as eye drops, at concentration of 100μg/ml.

Aldina et al (EurAsian J. of Biosci 13, 823-829 (2019) describe the anti-angiogenetic effect of a pure aqueous extract of Spirulina platensis by reducing the expression of the vascular Endothelial Growth Factor (VEGF) in corneal inflammation in rat. Among the known eye pathologies, there is the so called “dry eye” and it is also known that the contact lens wearing subjects often needs to moisturise the eye along the day. So, there is a sustained need to provide compositions for topical ophthalmic application which are effective and safe.

### OBJECT OF THE INVENTION

An object of the present invention is to provide a clear liquid ophthalmic composition comprising a full extract from Spirulina platensis.

A further object of the invention is the use of said clear liquid ophthalmic compositions in the eye pathologies and/or as eye drops and/or as artificial tears to be used in several eye pathologies, for example in the treatment of the eye dry symptoms as pain, hitch, ocular burning or the presence of foreign particles in the eyes. Such symptoms can be due to different origins, for example can be due to external factors, like air conditioning, pollution, air travels, work on video terminals, refractive surgery, contact lens wearing, of from pathologies, like the Meibomio gland disfunction, etc.

Another object of the present invention is a composition comprising a Spirulina platensis aqueous extract with increased viscosity.

A further object of the invention is to provide the use of said Spirulina platensis aqueous extract composition in various ophthalmic pathologies, such as the above defined ones.

### DESCRIPTION OF THE INVENTION

According to one of its aspects, a subject-matter of the present invention is a clear liquid ophthalmic composition comprising a Spirulina platensis full extract, and at least one liquid carrier suitable for ophthalmic use.

Spirulina platensis (here also referred to as “microorganism” or only Spirulina) is known in the art.

The expressions “ophthalmic composition” and “suitable for ophthalmic use” and similar expressions, mean that such a composition is suitable for eye administration and does not cause damages or side effects.

The expression “Spirulina platensis full extract”, or similar expressions herein means that said extract comprises all the different components of Spirulina platensis, in particular:
i. an “aqueous extract” which includes proteins, among them phycocyanins, water-soluble vitamins, amino acids and minerals, preferably a concentrated aqueous extract;
ii. a “lipidic soluble extract” which includes liposoluble vitamins, liposoluble amino acids and lipids, preferably a concentrated lipidic soluble extract; and
iii. a high molecular weight component extract which includes polysaccharides, preferably a concentrated high molecular weight component extract.

The above components (i) to (iii) are separately herein referred to as “pure concentrated fractions” or “pure concentrated extracts”.

A mixture of the above components (i) to (iii) is herein referred to as “concentrated (Spirulina platensis) full extract fractions” or “concentrated (Spirulina platensis) full extract extracts”.

This concentrated (Spirulina platensis) full extract, diluted with a suitable liquid carrier, provides the Spirulina platensis full extract composition of the invention.

The term “clear” herein means transparent, not-cloudy.

So, a subject-matter of the present invention is an ophthalmic composition comprising the “aqueous extract”, the “lipidic soluble extract” and “high molecular weight component extract” (polysaccharides) as above defined, i.e. the concentrated (Spirulina platensis) full extract and at least one liquid carrier suitable for ophthalmic use.

CN101444480 describes the use of the pure polysaccharide fraction (as (iii) above) for treatment of neovascular diseases. Aldina et al. (supra), discloses the use of the pure aqueous extract as an anti-inflammatory agent, i.e. an aqueous extract containing only water-soluble substances. Said documents however only disclose experimentations on animal models and are silent about pharmaceutical compositions. Indeed, both documents administer to animals eye drops comprising pure Spirulina extracts, without the addition of any other excipient, for instance buffers or agents suitable for increasing of the viscosity in the final solution.

According to a preferred embodiment, the composition comprising the Spirulina platensis full extract of the invention also comprises at least one solubilising agent such as, for example but not limited to, one or more cyclodextrins. According to a more preferred embodiment, said one or more cyclodextrins is hydroxypropyl-β-cyclodextrin.

So, contrary to the pure separate extracts of the prior art, the clear liquid full extract of Spirulina platensis of the invention provides improved performances thanks to the presence of the whole content of Spirulina. Indeed, in order to provide all the beneficial fractions of Spirulina, Applicant decided to separately extract the different fractions and then combine them in order to enjoy the advantages of the whole Spirulina content. However, Applicant observed that, due to the poor water solubility of the lipidic extract components (for example lipids or liposoluble vitamins), the full extract of Spirulina platensis is not a clear solution rather a suspension; as it is well known, substances which are not dissolved are less bioavailable. Thus, in order to overcome this problem, Applicant decided to try to add solubilising agents like, for example but not limited to, cyclodextrins, and obtained a clear solution.

Some techniques for the preparation of aqueous extracts of Spirulina platensis are known to the skilled in the art.

For example, a concentrated aqueous extract can be obtained subjecting a biomass, i.e. the whole alga, containing Spirulina platensis to a thermic cycle which includes: an initial storage of the biomass from −10 to −30° C., preferably −20° C., for some time, for instance about 10 days and a subsequent heating, for example in oven at the temperature from 45 to 55° C., preferably 50° C. for about some days, for instance 3 days. At the end of this cycle the biomass is filtered through a suitable filter, for example a 10 μm cellulose filter. The aqueous extract directly obtained by the above process is a “concentrated aqueous extract”, i.e. not yet diluted with water or other liquids, and is one of the active ingredients of the composition of the invention.

Also the lipidic soluble extract and the high molecular weight component extract including polysaccharides from Spirulina can be obtained with any technique known to the skilled in the art.

For example, the lipidic soluble extract can be obtained by treatment of the biomass with a suitable solvent, preferably a lower alcohol, such as ethanol, and subsequent filtration. The obtained lipidic soluble extract, is a concentrated lipophilic extract” is one of the active ingredients of the composition of the invention.

The high molecular weight component extract can be for example obtained from the biomass after a treatment with water at 90-120° C., preferably at 100° C. for some time, for instance for 1.5-3 hours, preferably 2 hours, followed by precipitation by the addition of 1% cetyltrimethylammonium bromide (CTAB) in water, at a temperature from 0 to 6° C., preferably 4° C., and a subsequent washing of the precipitate with a mixture of suitable solvents or liquid mixtures, such as sodium acetate and ethanol (for instance in a ratio 10/90 weight/volume). The high molecular weight component extract obtained as above is a concentrated high molecular weight component extract and is one of the active ingredients of the composition of the invention.

Some examples of the extraction processes are reported in the Experimental Section which follows for illustrative purposes only.

According to a preferred embodiment, such clear full extract is the main active ingredient included in the composition of the invention as above defined; however, if desired or needed, other active ingredients suitable for ophthalmic applications can be added to said composition.

For the preparation of the composition of the invention, in the form of eye drops, the concentrated extracts above mixed together to provide a concentrated Spirulina platensis full extract, which is subsequently diluted with a liquid carrier suitable for an ophthalmic administration.

For instance, for the administration, the concentrated Spirulina platensis full extract is diluted from 30 to 100 times (volume/volume), preferably from 40 to 60 times, with a liquid carrier suitable to an ophthalmic administration, preferably having a pH from 6.5 to 7.5, so that the concentrations of each concentrated extract, as above defined, in the final composition of the invention is about 0.05 to 0.2% weight/volume of the total composition.

Such liquid carrier can be made, for example, of a buffer solution conventionally used in ophthalmic area, preferably borate or phosphate buffers, boric acid or sodium borate/boric acid. The solution buffered, diluted so obtained, that constitutes the ophthalmic composition of the invention as above defined, can be filled in mono-dose or multi-dose devices suitable for the ophthalmic administration.

According to a preferred embodiment, the Spirulina platensis full extract composition of the invention also comprises one or more solubilising agents, such as, but not limited to, one or more cyclodextrins, preferably hydroxypropyl-β-cyclodextrin. Said one or more solubilising agents will be added in an amount from 0.05 to 0.5% by weight with respect to the total volume of the composition or, preferably, until the solution becomes clear.

According to a preferred embodiment, the Spirulina platensis full extract composition of the invention also comprises one or more viscosity enhancing agents, such as but not limited to carboxymethylcellulose.

The Spirulina platensis full extract composition is preferably characterised by a dynamic viscosity range from 1 to 20 mPa·s, preferably 5 mPa·s at 20° C. (detail in the Experimental Section).

The Spirulina platensis full extract composition can be used in the treatment of several eye pathologies, for example as artificial tears to overcome the dry eye syndrome, as moisturiser and/or to relief the symptoms derived from the poor presence of liquid in the conjunctive in humans and animals. Those symptoms can be due to any origin, for example from external factors, like air conditioning, pollution, air travel, video terminal work, refractive surgery, contact lens wearing, or from pathologies as the Meibomio gland disfunction, etc.

The Spirulina platensis full extract composition is also useful to the subjects wearing contact lenses.

According to another of its aspects, the present invention also relates to a liquid viscous composition comprising a pure Spirulina platensis aqueous extract as above defined, at least one viscosity enhancing agent and at least one liquid carrier suitable for ophthalmic use.

As stated above, the expression “pure Spirulina platensis aqueous extract” herein indicates an aqueous extract comprising the low molecular weight water extractable components of Spirulina platensis, in particular it includes proteins, among them the phytocyanins, vitamins, amino acids and minerals. Said pure Spirulina platensis aqueous extract may be obtained in the form of a concentrated extract as disclosed above.

As “viscosity enhancing agent” is herein meant excipient which are able to make a liquid composition more viscous, such as, but not limited to, carboxymethylcellulose (CMC), hydroxyethylcellulose (HEC) and hydroxypropylcellulose (HPC), CMC being preferred.

Said liquid carrier can be made, for example, by a buffer solution conventionally used in ophthalmic area, preferably borate or phosphate buffers, boric acid or sodium borate/boric acid. The solution buffered and diluted so obtained, that constitutes the ophthalmic composition of the invention, can be filled in mono-dose or multi-dose devices suitable for the ophthalmic administration.

So, for its administration, the concentrated Spirulina platensis aqueous extract is diluted from 30 to 100 times (volume/volume), preferably from 40 to 60 times, with a liquid carrier suitable to an ophthalmic administration, preferably having a pH from 6.5 to 7.5.

The viscous liquid composition comprising the pure aqueous extract of the invention provides an improved effectiveness and compliance when used as eye drop with respect to a non-viscous liquid composition. Indeed, the enhanced viscosity allows the solution to stay in the cornea for a longer time with respect to a non-viscous solution thanks to the higher resistance to the physiological eye rinse.

According to a preferred embodiment, the liquid viscous composition comprising of the invention comprising only the Spirulina platensis aqueous extract as the active ingredient, is added with a viscosity enhancer to provide a solution with a viscosity from 1 to 20 mPa·s measured by a rotational viscometer at 20° C. (details in the Experimental Section).

The liquid viscous composition comprising a pure Spirulina platensis aqueous extract is also useful in the treatment of eye pathologies.

If desired or needed, both the compositions of the invention may comprise other active ingredients suitable to treat the ophthalmic pathologies, and/or one or more suitable ophthalmic suitable excipients, such as hyaluronic acid or omega 3 fatty acids.

According to another aspect, another subject-matter of the invention is the clear liquid Spirulina platensis full extract composition or of the liquid viscous composition comprising a pure Spirulina platensis aqueous extract for use in the treatment of eye pathologies and/or as eye drops and/or as artificial tears to be used for example in the treatment of the eye dry symptoms like pain, hitch, ocular burning or sensation of e foreign body in the eyes in a subject, preferably to a mammalian, more preferably to a human being.

According to another aspect, another subject-matter of the invention is a method for the treatment of eye pathologies, for example in the treatment of the eye dry symptoms like pain, hitch, ocular burning or sensation of e foreign body in the eyes, which comprises the administration of an effective amount of the clear liquid Spirulina platensis full extract composition or of the liquid viscous composition comprising a pure Spirulina platensis aqueous extract to a subject, preferably to a mammalian, more preferably to a human being.

Eye drops comprising the clear liquid Spirulina platensis full extract composition or of the liquid viscous composition comprising a pure Spirulina platensis aqueous extract are another subject-matter of the invention.

The invention will be here described in the Experimental Section below, in a non-limiting way.

### EXPERIMENTAL SECTION

In the examples below the viscosity is measured at 20° C. by a Brookfield viscometer Dy-II Pro equipped with a Small Sample Adapter spare part and SC4-18 Spindler at 0.1-200 rpm increasing up to reach torque above 10%.

### Example 1

### Preparation of a Clear Liquid Spirulina Platensis Full Extract Composition Preparation of Spirulina Platensis Aqueous Extract (A)

The Spirulina platensis is obtained undergoing the biomass in water at −20° C. temperature for 10 days, and a subsequent storage in oven at 50° C. for 3 days. At the end of the thermal cycle the biomass is filtered through a 10 μm cellulose filter.

### Preparation of Spirulina Platensis Lipophilic Extract (B)

The Spirulina platensis lipophilic fraction is obtained by ethanol extraction (1 volume for 24 hours) of the biomass and subsequent filtration.

### Preparation of Spirulina Platensis Polysaccharide Extract (C)

The polysaccharide fraction is obtained by a treatment of the biomass with pure water at 100° C. for 2 hours and subsequent precipitation with 1% cetyltrimethylammonium bromide (CTAB) at 4° C. and wash of the precipitate with a mixture of sodium acetate and ethanol (10/90 ratio weight/volume).

### Preparation of the Clear Liquid Spirulina Platensis Full Extract Composition

1 ml of the aqueous extract obtained as described above (A), is added to 49 ml of 0.08 M pH 7.5 borate buffer, in which 250 mg of carboxymethylcellulose were previously dissolved. A solution is so obtained with a light blue/light green colour, to which 50 mg of the Spirulina platensis lipophilic fraction obtained as described above (B) and 27 mg of the Spirulina platensis polysaccharide fraction as described above (C) are added. The addition of the last two fractions induces a formation of a cloudy suspension. The suspension is added with 100 mg of hydroxypropyl-β-cyclodextrin. With this last addition the solution become clear with a light blue/light green colour and a dynamic viscosity measured at 20° C. of 16.30 mPa·s.

### Example 2

### Preparation of a Clear Liquid Spirulina Platensis Full Extract Composition

0.5 ml of the aqueous extract obtained as described in example 1 (A), is added to 49 ml of 0.08 M pH 7.5 borate buffer, in which 100 mg of carboxymethylcellulose were previously dissolved. A solution is obtained with a light blue/light green colour, to which 50 mg of the Spirulina platensis lipophilic fraction as described in example 1 (B) and 27 mg of the Spirulina platensis polysaccharide fraction obtained as described in example 1 (C) are added. The addition of the last two fractions induces a formation of a cloudy suspension. The suspension is added with 56 mg of hydroxypropyl-β-cyclodextrin. With this last addition the solution becomes clear with a light blue/light green colour and a dynamic viscosity measured at 20° C. of 3.70 mPa·s.

### Example 3

### Preparation of a Liquid Viscous Composition Comprising a Pure Spirulina Platensis Aqueous Extract

1 ml of the aqueous extract obtained as described in example 1 (A) is added to 49 ml of 0.08 M pH 7.5 borate buffer, in which 118 mg of carboxymethylcellulose were previously dissolved. A solution is so obtained with a light blue/light green colour characterised with a dynamic viscosity measured at 20° C. of 3.75 mPa·s.

### Example 4

### Preparation of a Liquid Viscous Composition Comprising a Pure Spirulina Platensis Aqueous Extract

1 ml of the aqueous extract obtained as described in example 1 (A), is added to 49 ml of 0.08 M pH 7,5 borate buffer, in which 118 mg of carboxymethylcellulose were previously dissolved. A solution is so obtained with a light blue/light green colour characterised with a dynamic viscosity measured at 20° C. of 1.29 of mPa·s.

1. A clear liquid ophthalmic composition comprising a Spirulina platensis full extract, and at least one liquid carrier suitable for ophthalmic use.

2. The composition according to claim 1, comprising
i. an aqueous extract which includes proteins, among them phycocyanins, water-soluble vitamins, amino acids and minerals;
ii. a lipidic soluble extract which includes liposoluble vitamins, liposoluble amino acids and lipids; and
iii. a high molecular weight component extract which includes polysaccharides.

3. The composition according to claim 1, wherein said at least one liquid carrier is a liquid carrier having a pH from 6.5 to 7.5.

4. The composition according to claim 3, wherein said at least one carrier is selected from borate or phosphate buffers, boric acid or sodium borate/boric acid.

5. The composition according to claim 1, further comprising at least one solubilising agent.

6. The composition according to claim 5, wherein said solubilising agent is a cyclodextrin.

7. The composition according to claim 2, wherein the concentration of each extract is 0.05 to 0.2% weight/volume of the total composition.

8. (canceled)

9. The composition according to claim 1 characterized by a dynamic viscosity of 1 to 20 mPa·s, at 20° C.

10. The composition according to claim 1, wherein said composition is viscous.

11. The composition according to claim 10, wherein said viscosity enhancing agent is selected from carboxymethylcellulose (CMC), hydroxyethylcellulose (HEC) and hydroxypropylcellulose (HPC), preferably carboxymethylcellulose (CMC).

12. The composition according to claim 10, wherein said at least one liquid carrier is a liquid carrier having a pH from 6.5 to 7.5.

13. The composition according to claim 12, wherein said at least one carrier is selected from borate or phosphate buffers, boric acid or sodium borate/boric acid.

14. The composition according to claim 10, wherein the concentration of the aqueous extract is 0.05 to 0.2% weight/volume of the total composition.

15. (canceled)

16. The composition according to claim 10, characterized by a dynamic viscosity from 1 to 20 mPa·s, at 20° C.

17. A method of treating eye pathologies with the composition of claim 1 as artificial tears to overcome dry eye syndrome or as moisturizer to relieve the symptoms derived from poor presence of liquid in the conjunctive in humans or animals, said method comprising:
applying said composition to said humans and animals and
treating said eye pathologies in said humans or animals.

18. A method of treating eye pathologies with the composition of claim 10 as artificial tears to overcome dry eye syndrome or as moisturizer to relieve the symptoms derived from poor presence of liquid in the conjunctive in humans or animals, said method comprising:
applying said composition to said humans and animals and
treating said eye pathologies in said humans or animals.
